(19) United States
(12) Patent Application Publication
(10) Pub. No.: US 2008/0167693 A1
(43) Pub. Date: Jul. 10, 2008

(54) METHOD AND APPARATUS FOR SYNCHRONIZING NEURAL STIMULATION TO CARDIAC CYCLES

(75) Inventors: Robert S. Kieval, Medina, MN (US); Martin A. Rossing, Coon Rapids, MN (US); Eric D. Irwin, Minneapolis, MN (US); John R. Brintnall, Bloomington, MN (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: CVRx, Inc., Minneapolis, MN (US)

(21) Appl. No.: 11/868,408

(22) Filed: Oct. 5, 2007

Related U.S. Application Data
(63) Continuation of application No. 10/958,694, filed on Oct. 4, 2004.

(51) Int. Cl. A61N 1/365

(52) U.S. Cl. 607/18

(57) ABSTRACT

The present invention comprises a neural stimulator having an output to deliver neural stimulation pulses, a sensor to sense a reference signal indicative of cardiac cycles each including a predetermined type timing reference event, the sensor being external to the circulatory system. The neural stimulator further includes detection circuitry coupled to the sensor to detect the predetermined type timing reference event, and a control circuit having an offset interval generator and a pulse delivery controller.

### CROSS-REFERENCES TO RELATED APPLICATIONS

This application is a continuation of U.S. patent application Ser. No. 10/958,694, filed on Oct. 4, 2004, the full disclosure of which is hereby incorporated by reference. This application is related to but does not claim the benefit of U.S. Pat. No. 6,522,926, filed on Sep. 27, 2000, U.S. Pat. No. 6,616,624, filed on Oct. 30, 2000, U.S. Pat. No. 6,985,774, filed on Sep. 26, 2001, U.S. Pat. No. 6,850,801, filed on Sep. 26, 2001, and U.S. Pat. No. 7,158,832, filed Sep. 26, 2001, all of which are hereby fully incorporated by reference. This application is also related to PCT Patent Application No. PCT/US01/30249, filed Sep. 27, 2001, and the following U.S. patent applications, all of which are hereby incorporated fully by reference: Ser. No. 10/284,063 filed Oct. 29, 2002; Ser. No. 10/453,678 filed Jun. 2, 2003; Ser. No. 10/402,911 filed Mar. 27, 2003; Ser. No. 10/402,393 filed Mar. 27, 2003; Ser. No. 10/818,738 filed Apr. 5, 2004; and Ser. No. 60/584,730 filed Jun. 30, 2004.

### BACKGROUND OF THE INVENTION

1. Field of the Invention

The present invention relates generally to medical devices and methods for treating heart failure. More specifically, the present invention involves baroreflex activation and cardiac resynchronization to treat heart failure.

Congestive heart failure (CHF) is an imbalance in pump function in which the heart fails to maintain the circulation of blood adequately. The most severe manifestation of CHF, pulmonary edema, develops when this imbalance causes an increase in lung fluid due to leakage from pulmonary capillaries into the lung. More than 3 million people have CHF, and more than 400,000 new cases present yearly. Prevalence of CHF is 1-2% of the general population. Approximately 30-40% of patients with CHF are hospitalized every year. CHF is the leading diagnosis-related group (DRG) among hospitalized patients older than 65 years. The 5-year mortality rate after diagnosis of CHF is around 60% in men and 45% in women.

The most common cause of heart failure is coronary artery disease, which is secondary to loss of left ventricular muscle, ongoing ischemia, or decreased diastolic ventricular compliance. Other causes of CHF include hypertension, valvular heart disease, congenital heart disease, other cardiomyopathies, myocarditis, and infectious endocarditis. CHF often is precipitated by cardiac ischemia or arrhythmias, cardiac or extracardiac infection, pulmonary embolus, physical or environmental stresses, changes or noncompliance with medical therapy, dietary indiscretion, or iatrogenic volume overload.

A number of different treatment modalities may be attempted for treating heart failure, such as medications, mechanical restriction of the heart, surgical procedures to reduce the size of an expanded heart and the like. One preferred heart failure treatment method is cardiac resynchronization therapy (CRT). CRT uses a pacemaker with multiple pacing leads to coordinate the heart's four chambers to act together in a sequence that will pump blood more efficiently. CRT generally improves the pumping efficiency of the heart by providing an electrical stimulation to a later-contracting chamber, or to a later-contracting chamber portion (e.g., the left ventricle free wall) contemporaneously with the natural contraction of the earlier contracting portion, such as the septum. Because adjacent chambers and/or both walls of a ventricle contract at approximately the same time with CRT, the pumping efficiency of the heart may be significantly improved. Although CRT may sometimes provide effective treatment of CHF, in some cases CRT alone only acts as a temporary or incomplete treatment. Used by itself, CRT may also lead to one or more side effects, such as cardiac arrhythmia.

Another CHF treatment method that has been proposed is to affect the baroreflex system to help the heart perform more efficiently. Baroreflex activation may generally decrease neurohormonal activation, thus decreasing cardiac afterload, heart rate, sympathetic drive to the heart and the like. By decreasing the demands placed on the heart, baroreflex activation may help prevent or treat CHF.

Treating underlying cardiac arrhythmias is another possible strategy for preventing or treating CHF. Pacemaker devices, for example, may be used to treat an arrhythmia. Alternatively or additionally, baroreflex activation may be used to treat a cardiac arrhythmia. Methods and devices for such baroreflex activation for arrhythmia treatment are described, for example, in U.S. Patent Application No. 60/584,730, which was previously incorporated by reference.

Of course, no “perfect” treatment method for heart failure has yet been developed. Although some of the therapies mentioned above may be highly effective in some cases, some may have unwanted side effects or provide little benefit to some patients. Because CHF is such a pervasive health problem, with high morbidity, mortality and costs to society, improved treatment methods are continually sought.

Therefore, it would be desirable to provide improved methods and apparatus for treating heart failure. Ideally, such methods and apparatus would be minimally invasive, with few if any significant side effects. Ideally, one or more underlying mechanisms causing heart failure could be treated in some cases. At least some of these objectives will be met by the present invention.

2. Description of the Background Art

Rau et al. (2001) Biological Psychology 57:179-201 describes animal and human experiments involving baroreceptor stimulation. U.S. Pat. Nos. 6,073,048 and 6,178,349, each having a common inventor with the present application, describe the stimulation of nerves to regulate the heart, vasculature, and other body systems. U.S. Pat. No. 6,522,926, assigned to the assignee of the present application, describes activation of baroreceptors by multiple modalities. Nerve stimulation for other purposes is described in, for example, U.S. Pat. Nos. 6,292,695 B1 and 5,700,282. Publications which describe the existence of baroreceptors and/or related receptors in the venous vasculature and atria include Goldberger et al. (1999) J. Neuro. Meth. 91:109-114; Kostreva and Pontus (1993) Am. J. Physiol. 265:G15-G20; Coleridge et al. (1973) Circ. Res. 23:87-97; Mifflin and Kunze (1982) Circ. Res. 51:241-249; and Schaurte et al. (2000) J. Cardiovasc Electrophysiol. 11:64-69. U.S. Pat. No. 5,203,326 describes an anti-arrhythmia pacemaker. PCT patent application publication number WO 99/51286 describes a system for regulating blood flow to a portion of the vasculature to treat heart disease. The full texts and disclosures of all the references listed above are hereby incorporated fully by reference.

Cardiac resynchronization therapy (CRT) devices are known. Examples of CRT devices and methods are described in U.S. Pat. Nos. 6,768,923; 6,766,189; 6,748,272; 6,704,598; 6,701,186; and 6,666,826, the full disclosures of which are hereby incorporated by reference.

### BRIEF SUMMARY OF THE INVENTION

In one aspect of the present invention, a method for treating heart failure in a patient involves activating a baroreflex system of the patient with at least one baroreflex activation device and resynchronizing the patient's heart with a cardiac resynchronization device. Activating the patient's baroreflex system may improve the efficiency of the heart, by reducing afterload, heart rate, sympathetic drive to the heart and/or the like. Cardiac resynchronization therapy (CRT) additionally promotes efficiency of the heart by synchronizing contractions of the heart chambers. In some embodiments, both baroreflex activation and resynchronization are performed by one combined implantable device.

In some embodiments, the activating and resynchronizing steps are performed simultaneously. Alternatively, the activating and resynchronizing steps may be performed sequentially. Generally, any of a number of suitable anatomical structures may be activated to provide baroreflex activation. For example, in various embodiments, activating the baroreflex system may involve activating one or more baroreceptors, one or more nerves coupled with a baroreceptor, a carotid sinus nerve, or some combination thereof. In embodiments where one or more baroreceptors are activated, the baroreceptor(s) may sometimes be located in arterial vasculature, such as but not limited to a carotid sinus, aortic arch, heart, common carotid artery, subclavian artery, pulmonary artery, femoral artery and/or brachiocephalic artery. Alternatively, a baroreflex activation device may be positioned in the low-pressure side of the heart or vasculature, as described in U.S. patent application Ser. No. 10/284,063, previously incorporated by reference, in locations such as an inferior vena cava, superior vena cava, portal vein, jugular vein, subclavian vein, iliac vein, azygous vein, pulmonary vein and/or femoral vein. In many embodiments, the baroreflex activation device is implanted in the patient. The baroreflex activation may be achieved, in various embodiments, by electrical activation, mechanical activation, thermal activation and/or chemical activation. Furthermore, baroreflex activation may be continuous, pulsed, periodic or some combination thereof in various embodiments.

Optionally, the method may further involve sensing a patient condition and modifying baroreflex activation and/or resynchronization based on the sensed patient condition. For example, sensing the patient condition may involve sensing physiological activity with one or more sensors. Sensors, may include an extracardiac electrocardiogram (ECG), an intracardiac ECG, an impedance sensor, a volume sensor, an implantable pressure sensor, an accelerometer, an edema sensor, any combination of these sensors, or any other suitable sensors or combinations of sensors. The sensed patient condition may comprise any of a number of suitable physiological conditions in various embodiments, such as but not limited to a change in heart rate, a change in relative timing of atrial and/or ventricular contractions, a change in a T-wave and/or S-T segment on an ECG, presence of edema and/or the like. Generally, any suitable data may be acquired by one or more sensors. In one embodiment, for example, sensing involves acquiring pressure data from the patient's heart. Such pressure data may then be converted into cardiac performance data. Thus, some embodiments further include processing one or more sensed conditions into data and optionally providing the data to the baroreflex activation device and/or the resynchronization device.

In some embodiments, resynchronizing involves delivering a stimulus to the heart to cause at least a portion of the heart to contract. Optionally, the method may further include, before and/or during resynchronization, sensing a cardiac event in at least a portion of the heart. For example, the cardiac event may comprise a contraction, an electrical contraction signal originating in the heart, an electrical pacemaker signal, or the like. In some embodiments, resynchronization further involves preventing or distinguishing sensation of an activation signal from the baroreflex activation device. In other words, the sensor (or a processor coupled with the sensor) may be adapted to sense one or more cardiac events or parameters while ignoring (or filtering out) signals emitted from the baroreflex activation device. In various embodiments, the cardiac event is sensed in one of a number of different portions of the heart, and the stimulus is delivered to that portion and/or to another portion. For example, in one embodiment, the cardiac event is sensed on one side of the heart, and the stimulus is delivered to that side and/or to the opposite side. In some embodiments, the cardiac event is sensed in one or more heart chambers, and the stimulus is delivered to one or more chambers. In some embodiments, for example, the event is sensed in one or more atria of the heart and the stimulus is delivered to one or more ventricles. In other embodiments, sensing and stimulus delivery are performed in only ventricles or only atria. Any suitable combination of sensing area(s) and stimulus delivery area(s) are contemplated.

In addition to resynchronization therapy, in some embodiment, the method further includes applying therapy directed at preventing and/or treating a cardiac arrhythmia. Such therapy may be applied, for example, via a cardiac pacemaker or a combined pacemaker/defibrillator. The pacemaker component of the device, in some embodiments, may be a biventricular pacemaker.

In another aspect of the invention, a method for treating heart failure in a patient involves sensing at least one patient condition, activating a baroreflex system of the patient with at least one baroreflex activation device, and resynchronizing the patient's heart with a cardiac resynchronization device. In this method, at least one of the activating and resynchronizing steps is based at least partially on the sensed patient condition. Any features of the methods described above may be applied.

In another aspect of the present invention, a device for treating heart failure in a patient includes at least one baroreflex activation member and at least one cardiac resynchronization member coupled with the baroreflex activation member. In some embodiments, the device is implantable within the patient. Optionally, the device may also include at least one sensor coupled with the device for sensing one or more patient conditions. Such a device may further include a processor coupled with the sensor for processing the sensed patient condition(s) into data and providing the data to the baroreflex activation member(s) and/or the cardiac resynchronization member(s). In some embodiments, the processor is adapted to distinguish the sensed patient condition(s) from one or more signals transmitted from the baroreflex activation member(s).

In some embodiments, the device includes at least one physiological sensor. For example, the sensor may include, but is not limited to, an electrocardiogram, a pressure sensing device, a volume sensing device, an accelerometer or an edema sensor. In various embodiments, sensor(s) may be adapted to sense heart rate, cardiac waveform, timing of atrial and/or ventricular contractions, venous or arterial pressure, venous or arterial volume, cardiac output, pressure and/or volume in one or more heart chambers, cardiac efficiency, cardiac impedance, edema and/or the like.

In some embodiments, the resynchronization member comprises a cardiac pacemaker. For example, in a number of embodiments, the pacemaker comprises a biventricular pacemaker. Such a resynchronization member may also be used to prevent and/or treat cardiac arrhythmias. To that end, in one embodiment, the resynchronization member may comprise a combined pacemaker/defibrillator.

In another aspect of the present invention, a system for treating heart failure in a patient includes: at least one baroreflex activation device; at least one cardiac resynchronization device coupled with the baroreflex activation device; and at least one sensor coupled with the cardiac resynchronization device for sensing one or more patient conditions. In some embodiments, the entire system is implantable within the patient, while in other embodiments only part of the system is implantable and the remainder of the system resides outside the patient. Optionally, the system may further include a processor coupled with the sensor for processing the sensed patient condition(s) into data and providing the data to one or more baroreflex activation devices and one or more cardiac resynchronization devices. Any features of the baroreflex activation and resynchronization members described above may be applied to the baroreflex activation and resynchronization devices of the system, in various embodiments.

These and other aspects and embodiments of the present invention are described in further detail below, with reference to the attached drawings.

### BRIEF DESCRIPTION OF THE DRAWINGS

FIG. 1 is a schematic illustration of the upper torso of a human body showing the major arteries and veins and associated anatomy;

FIG. 2A is a cross sectional schematic illustration of a carotid sinus and baroreceptors within a vascular wall;

FIG. 2B is a schematic illustration of baroreceptors within a vascular wall and the baroreflex system;

FIG. 3 is a block diagram of a baroreflex activation and cardiac resynchronization therapy system for treating heart failure according to one embodiment of the present invention;

FIG. 4 is a flow diagram of a baroreflex activation and cardiac resynchronization therapy system for treating heart failure according to one embodiment of the present invention; and

FIGS. 5A and 5B are schematic illustrations of a baroreflex activation device in the form of an internal, inflatable, helical balloon, stent or coil, which mechanically induces a baroreflex signal in accordance with an embodiment of the present invention.

### DETAILED DESCRIPTION OF THE INVENTION

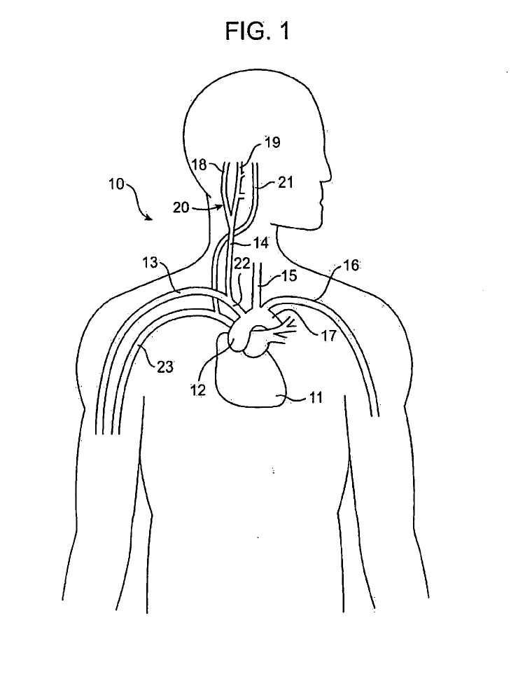

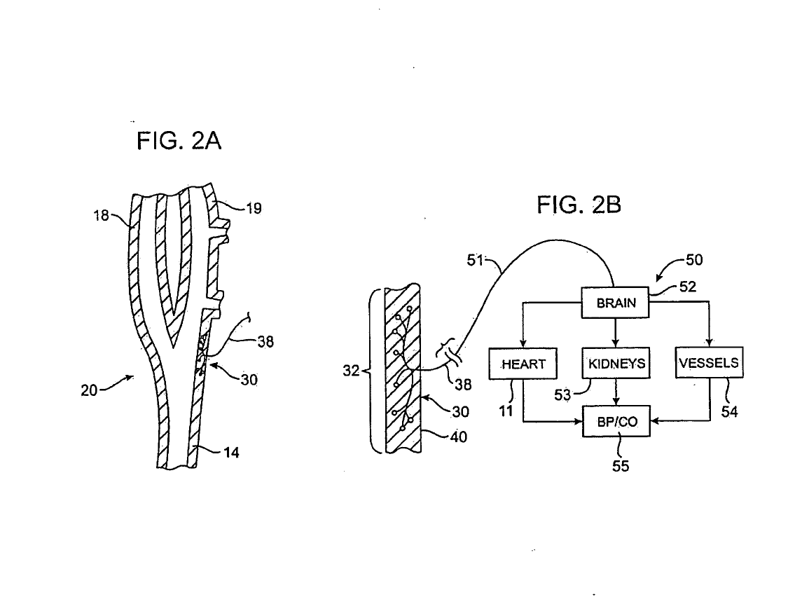

Referring now to FIGS. 1, 2A and 2B, within the arterial walls of the aortic arch 12, common carotid arteries 14/15 (near the right carotid sinus 20 and left carotid sinus), subclavian arteries 13/16 and brachiocephalic artery 22 there are baroreceptors 30. For example, as best seen in FIG. 2A, baroreceptors 30 reside within the vascular walls of the carotid sinus 20. Baroreceptors 30 are a type of stretch receptor used by the body to sense blood pressure. An increase in blood pressure causes the arterial wall to stretch, and a decrease in blood pressure causes the arterial wall to return to its original size. Such a cycle is repeated with each beat of the heart. Baroreceptors 30 located in the right carotid sinus 20, the left carotid sinus and the aortic arch 12 play the most significant role in sensing blood pressure that affects baroreflex system 50, which is described in more detail with reference to FIG. 2B.

With reference now to FIG. 2B, a schematic illustration shows baroreceptors 30 disposed in a generic vascular wall 40 and a schematic flow chart of baroreflex system 50. Baroreceptors 30 are profusely distributed within the arterial walls 40 of the major arteries discussed previously, and generally form an arbor 32. The baroreceptor arbor 32 comprises a plurality of baroreceptors 30, each of which transmits baroreceptor signals to the brain 52 via nerve 38. Baroreceptors 30 are so profusely distributed and arborized within the vascular wall 40 that discrete baroreceptor arbors 32 are not readily discernable. To this end, baroreceptors 30 shown in FIG. 2B are primarily schematic for purposes of illustration.

In addition to baroreceptors, other nervous system tissues are capable of inducing baroreflex activation. For example, baroreflex activation may be achieved in various embodiments by activating one or more baroreceptors, one or more nerves coupled with one or more baroreceptors, a carotid sinus nerve or some combination thereof. Therefore, the phrase “baroreflex activation” generally refers to activation of the baroreflex system by any means, and is not limited to directly activating baroreceptor(s). Although the following description often focuses on baroreflex activation/stimulation and induction of baroreceptor signals, various embodiments of the present invention may alternatively achieve baroreflex activation by activating any other suitable tissue or structure. Thus, the terms “baroreflex activation device” and “baroreflex activation device” are used interchangeably in this application.

Baroreflex signals are used to activate a number of body systems which collectively may be referred to as baroreflex system 50. Baroreceptors 30 are connected to the brain 52 via the nervous system 51, which then activates a number of body systems, including the heart 11, kidneys 53, vessels 54, and other organs/tissues via neurohormonal activity. Although such activation of baroreflex system 50 has been the subject of other patent applications by the inventors of the present invention, the focus of the present invention is the effect of baroreflex activation on the brain 52 to prevent cardiac arrhythmias and/or promote recovery after occurrence of an arrhythmia.

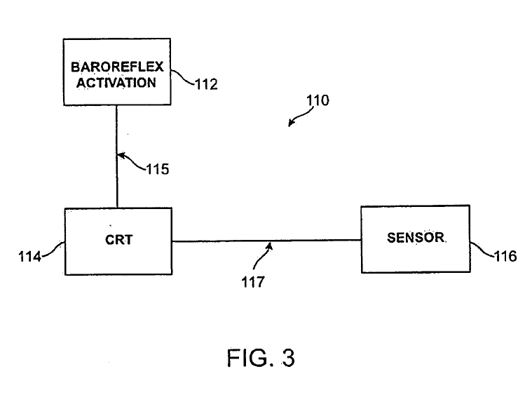

With reference to FIG. 3, in one embodiment a heart failure treatment system 110 includes a baroreflex activation device 112, a cardiac resynchronization therapy (CRT) device 114 and one or more sensors 116. In one embodiment, the baroreflex activation device 112 is coupled with the CRT device 114 via a cable 115, though any other suitable connection means may be used in alternate embodiments. The CRT device 114 may likewise be coupled with the sensor 116 via a cable 117 or any other suitable means. In various alternative embodiments, the sensor 116 (or multiple sensors) may be coupled directly with the baroreflex activation device 112 or with both the activation device 112 and the CRT device 114. In an alternative embodiment, the baroreflex activation device 112 and the CRT device 114 may be combined into on unitary device, with the unitary device being coupled with one or more sensors. In yet another embodiment, the unitary device may also be combined with one or more built-in sensors 116.

CRT devices 114 are known in the art, and any suitable CRT device 114 now known or hereafter developed may be used in various embodiments of the present invention. For example, the CRT device 114 may be the same as or similar to those described in U.S. Pat. Nos. 6,768,923; 6,766,189; 6,748,272; 6,704,598; 6,701,186, and 6,666,826, which were previously incorporated by reference. Alternatively, any other suitable CRT device 114 may be incorporated into the heart failure treatment system 110. In some embodiments, CRT device 114 may comprise a combined pacemaker/defibrillator, and in some cases a biventricular pacemaker/defibrillator.

Any suitable baroreflex activation device 112 (or multiple devices) may also be used, in various embodiments. Examples of suitable baroreflex activation devices 112 include, but are not limited to, those described in detail in U.S. Pat. Nos. 6,522,926 and 6,616,624, and U.S. patent application Ser. Nos. 09/964,079, 09/963,777, 09/963,991, 10/284,063, 10/453,678, 10/402,911, 10/402,393, 10/818,738, and 60/584,730, which were previously incorporated by reference. Any number or type of suitable baroreflex activation device 112 may be used, in accordance with various embodiments, and the activation device(s) 112 may be placed in any suitable anatomical location. For further details regarding specific exemplary baroreflex activation devices 112, reference may be made to any of the patents or patent applications listed immediately above.

The sensor 116 (or in some embodiments multiple sensors) may include any suitable sensor device or combination of devices. Oftentimes, the sensor(s) 116 is adapted for positioning in or on the heart 11, although in various alternative embodiments sensor(s) 116 may be placed in one or more blood vessels, subcutaneously, in any other suitable location in the patient, or even outside the patient, such as with an external electrocardiogram device. Examples of sensors 116 include, but are not limited to, electrocardiogram devices, pressure sensors, volume sensors, accelerometers, edema sensors and/or the like. Sensor(s) 116 may sense any suitable patient characteristic (or condition), such as but not limited to heart rate, cardiac waveform, timing of atrial and/or ventricular contractions, venous or arterial pressure, venous or arterial volume, cardiac output, pressure and/or volume in one or more heart chambers, cardiac efficiency, cardiac impedance and/or edema. Again, in various embodiments any suitable sensor device(s) 116 may be used and any suitable condition may be sensed.

Generally, the sensor 116 may provide information about sensed patient conditions either to the CRT device 114, the baroreflex activation device 112, or both. In some embodiments, such information may then be used by the CRT device 114 and/or the baroreflex activation device 112 to either initiate or modify a treatment. Typically, though not necessarily, the system 110 includes a processor for converting sensed information into data that is usable by the CRT device 114 and/or the baroreflex activation device 112. Such a processor is described in further detail below.

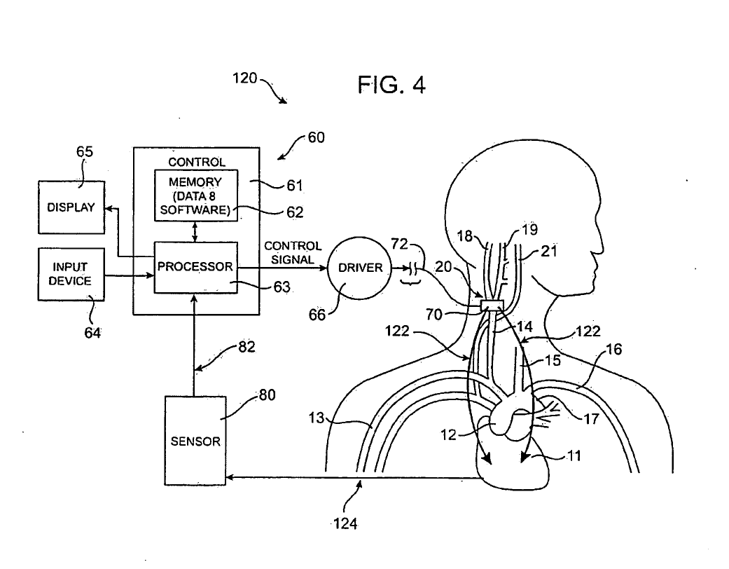

Referring now to FIG. 4, another embodiment of a heart failure treatment system 120 is shown in the form of a flow diagram. In this embodiment, the system 120 includes a processor 63, a combined baroreflex activation/CRT device 70, and a sensor 80. For clarity, the sensor 80 is shown as one unit located outside the patient, such as would be the case if the sensor 80 comprised an external electrocardiogram (ECG) device. In alternative embodiments, however, the sensor 80 (or multiple sensors) may be located on or in the heart 11 or in any other suitable location within the patient. Optionally, processor 63 may be part of a control system 60, which may include a control block 61 (housing processor 63 and memory 62), a display 65 and/or and input device 64. Processor 63 is coupled with sensor 80 by an electric sensor cable or lead 82 and to baroreflex/CRT device 70 by an electric control cable 72. (In alternative embodiments, lead 82 may be any suitable corded or remote connection means, such as a remote signaling device.) Thus, processor 63 receives a sensor signal from sensor 80 by way of sensor lead 82 and transmits a control signal to baroreflex/CRT device 70 by way of control cable 72. In an alternative embodiment, the processor 63 may be combined in one unitary device with the baroreflex/CRT device 70.

As discussed above, the CRT component of the baroreflex/CRT device 70 may be any suitable CRT device. Generally, the combined device 70 includes one or more pacing leads 122 for coupling the device 70 with the heart 11. In one embodiment, for example, the device 70 includes two pacing leads 122 for providing biventricular pacing. Generally, the heart 11 may be coupled with the sensor 80 one or more leads 124, such as with an ECG device. In other embodiments, the sensor(s) 80 may be attached directly to a wall of the heart 11 or to any other suitable anatomical structure.

As mentioned above, the sensor 80 generally senses and/or monitors one or more parameters, such as but not limited to change in heart rate, change in cardiac pressure(s), change in contraction timing of one or both atria and ventricles of the heart, change in electrocardiogram shape (such as T-wave shape), change in blood pressure and/or the like. The parameter sensed by sensor 80 is then transmitted to processor 63, which may generate a control signal as a function of the received sensor signal. A control signal will typically be generated, for example, when a sensor signal is determined to be indicative of heart failure or potentially ensuing heart failure. If decreased cardiac efficiency, for example, is determined to be an advance indicator of the onset of heart failure, data that is sensed and processed and determined to be indicative of decreased efficiency will cause processor 63 to generate a control signal. The control signal activates, deactivates, modifies the intensity or timing of, or otherwise modulates baroreflex/CRT device 70. In some embodiments, for example, baroreflex/CRT device 70 may activate an ongoing baroreflex at a constant rate until it receives a control signal, which may cause the device 70 to either increase or decrease intensity of its baroreflex activation and/or alter its resynchronization timing in various embodiments. In another embodiment, baroreflex/CRT device 70 may remain in a turned-off mode until activated by a control signal from processor 63. In another embodiment, when sensor 80 detects a parameter indicative of normal body function (e.g., steady heart rate and/or steady intracardiac pressures), processor 63 generates a control signal to modulate (e.g., deactivate) baroreflex/CRT device 70. Any suitable combination is contemplated in various embodiments.

Again, sensor 80 may comprise any suitable device that measures or monitors a parameter indicative of the need to modify baroreflex activation and/or cardiac resynchronization. For example, sensor 80 may comprise a physiologic transducer or gauge that measures cardiac activity, such as an ECG. Alternatively, sensor 80 may measure cardiac activity by any other technique, such as by measuring changes in intracardiac pressures or the like. Examples of suitable transducers or gauges for sensor 80 include ECG electrodes and the like. Although only one sensor 80 is shown, multiple sensors 80 of the same or different type at the same or different locations may be utilized. Sensor 80 is preferably positioned on or near the patient's heart, one or near major vascular structures such as the thoracic aorta, or in another suitable location to measure cardiac activity, such as increased heart rate or pressure changes. Sensor 80 may be disposed either inside or outside the body in various embodiments, depending on the type of transducer or gauge utilized. Sensor 80 may be separate from baroreflex/CRT device 70, as shown schematically in FIG. 4, or may alternatively be combined therewith in one device.

The baroreflex activation component of the baroreflex/CRT device 70 may comprise a wide variety of devices which utilize mechanical, electrical, thermal, chemical, biological, or other means to activate baroreceptors 30 and/or other tissues. Specific embodiments of baroreflex/CRT device 70 are discussed, for example, in U.S. patent application Ser. Nos. 09/964,079, 09/963,777, 09/963,991, 10/284,063, 10/453,678, 10/402,911, 10/402,393, 10/818,738, and 60/584,730, which were previously incorporated by reference. In many embodiments, particularly the mechanical activation embodiments, the baroreflex/CRT device 70 indirectly activates one or more baroreceptors 30 by stretching or otherwise deforming the vascular wall 40 surrounding baroreceptors 30. In some other instances, particularly the non-mechanical activation embodiments, baroreflex/CRT device 70 may directly activate one or more baroreceptors 30 by changing the electrical, thermal or chemical environment or potential across baroreceptors 30. It is also possible that changing the electrical, thermal or chemical potential across the tissue surrounding baroreceptors 30 may cause the surrounding tissue to stretch or otherwise deform, thus mechanically activating baroreceptors 30. In other instances, particularly the biological activation embodiments, a change in the function or sensitivity of baroreceptors 30 may be induced by changing the biological activity in baroreceptors 30 and altering their intracellular makeup and function.

Many embodiments of the baroreflex/CRT device 70 are suitable for implantation, and are preferably implanted using a minimally invasive percutaneous translumenal approach and/or a minimally invasive surgical approach, depending on whether the device 70 is disposed intravascularly, extravascularly or within the vascular wall 40. The baroreflex/CRT device 70 may be positioned anywhere baroreceptors 30 affecting baroreflex system 50 are numerous, such as in the heart 11, in the aortic arch 12, in the common carotid arteries 18/19 near the carotid sinus 20, in the subclavian arteries 13/16, or in the brachiocephalic artery 22. The baroreflex/CRT device 70 may be implanted such that the device 70 is positioned immediately adjacent baroreceptors 30. Alternatively, the device 70 may be positioned in the low-pressure side of the heart or vasculature, near a baroreceptor, as described in U.S. patent application Ser. No. 10/284,063, previously incorporated by reference. In fact, the baroreflex/CRT device 70 may even be positioned outside the body such that the device 70 is positioned a short distance from but proximate to baroreceptors 30. In one embodiment, the baroreflex/CRT device 70 is implanted near the right carotid sinus 20 and/or the left carotid sinus (near the bifurcation of the common carotid artery) and/or the aortic arch 12, where baroreceptors 30 have a significant impact on baroreflex system 50. For purposes of illustration only, the present invention is described with reference to the baroreflex/CRT device 70 positioned near the carotid sinus 20.

Memory 62 may contain data related to the sensor signal, the control signal, and/or values and commands provided by input device 64. Memory 62 may also include software containing one or more algorithms defining one or more functions or relationships between the control signal and the sensor signal. The algorithm may dictate activation or deactivation control signals depending on the sensor signal or a mathematical derivative thereof. The algorithm may dictate an activation or deactivation control signal when the sensor signal falls below a lower predetermined threshold value, rises above an upper predetermined threshold value or when the sensor signal indicates a specific physiologic event.

As mentioned previously, the baroreflex/CRT device 70 may activate baroreceptors 30 mechanically, electrically, thermally, chemically, biologically or otherwise. In some instances, control system 60 includes a driver 66 to provide the desired power mode for the baroreflex/CRT device 70. For example if the baroreflex/CRT device 70 utilizes pneumatic or hydraulic actuation, driver 66 may comprise a pressure/vacuum source and the cable 72 may comprise fluid line(s). If the baroreflex/CRT device 70 utilizes electrical or thermal actuation, driver 66 may comprise a power amplifier or the like and the cable 72 may comprise electrical lead(s). If baroreflex/CRT device 70 utilizes chemical or biological actuation, driver 66 may comprise a fluid reservoir and a pressure/vacuum source, and cable 72 may comprise fluid line(s). In other instances, driver 66 may not be necessary, particularly if processor 63 generates a sufficiently strong electrical signal for low level electrical or thermal actuation of baroreflex/CRT device 70.

Control system 60 may operate as a closed loop utilizing feedback from sensor 80, or as an open loop utilizing commands received by input device 64. The open loop operation of control system 60 preferably utilizes some feedback from sensor 80, but may also operate without feedback. Commands received by the input device 64 may directly influence the control signal or may alter the software and related algorithms contained in memory 62. The patient and/or treating physician may provide commands to input device 64. Display 65 may be used to view the sensor signal, control signal and/or the software/data contained in memory 62.

The control signal generated by control system 60 may be continuous, periodic, episodic or a combination thereof, as dictated by an algorithm contained in memory 62. The algorithm contained in memory 62 defines a stimulus regimen which dictates the characteristics of the control signal as a function of time, and thus dictates baroreflex activation as a function of time. Continuous control signals include a pulse, a train of pulses, a triggered pulse and a triggered train of pulses, all of which are generated continuously. Examples of periodic control signals include each of the continuous control signals described above which have a designated start time (e.g., beginning of each minute, hour or day) and a designated duration (e.g., 1 second, 1 minute, 1 hour). Examples of episodic control signals include each of the continuous control signals described above which are triggered by an episode (e.g., activation by the patient/physician, an increase in blood pressure above a certain threshold, etc.).

The stimulus regimen governed by control system 60 may be selected to promote long term efficacy. It is theorized that uninterrupted or otherwise unchanging activation of baroreceptors 30 may result in the baroreceptors and/or the baroreflex system becoming less responsive over time, thereby diminishing the long-term effectiveness of the therapy. Therefore, the stimulus regimen may be selected to activate, deactivate or otherwise modulate baroreflex/CRT device 70 in such a way that therapeutic efficacy is maintained long term.

In addition to maintaining therapeutic efficacy over time, the stimulus regimens of the present invention may be selected to reduce power requirement/consumption of control system 60. As will be described in more detail, the stimulus regimen may dictate that baroreflex/CRT device 70 be initially activated at a relatively higher energy and/or power level, and subsequently activated at a relatively lower energy and/or power level. The first level attains the desired initial therapeutic effect, and the second (lower) level sustains the desired therapeutic effect long term. By reducing the energy and/or power level after the desired therapeutic effect is initially attained, the power required or consumed by the device 70 is also reduced long term. This may correlate into systems having greater longevity and/or reduced size (due to reductions in the size of the power supply and associated components).

Another advantage of the stimulus regimens of the present invention is the reduction of unwanted collateral tissue stimulation. As mentioned above, the stimulus regimen may dictate that baroreflex/CRT device 70 be initially activated at a relatively higher energy and/or power level to attain the desired effect, and subsequently activated at a relatively lower energy and/or power level to maintain the desired effect. By reducing the output energy and/or power level, the stimulus may not travel as far from the target site, thereby reducing the likelihood of inadvertently stimulating adjacent tissues such as muscles in the neck and head.

Such stimulus regimens may be applied to all baroreflex activation and cardiac resynchronization embodiments described herein. In addition to baroreflex/CRT devices 70, such stimulus regimens may be applied to the stimulation of the carotid sinus nerves or other nerves. In particular, the stimulus regimens described herein may be applied to baropacing (i.e., electrical stimulation of the carotid sinus nerve), as in the baropacing system disclosed in U.S. Pat. No. 6,073,048 to Kieval et al., the entire disclosure of which is incorporated herein by reference.

The stimulus regimen may be described in terms of the control signal and/or the output signal from baroreflex/CRT device 70. Generally speaking, changes in the control signal result in corresponding changes in the output of baroreflex/CRT device 70 which affect corresponding changes in baroreceptors 30. The correlation between changes in the control signal and changes in baroreflex/CRT device 70 may be proportional or disproportional, direct or indirect (inverse), or any other known or predictable mathematical relationship. For purposes of illustration only, the stimulus regimen may be described herein in such a way that assumes the output of baroreflex/CRT device 70 is directly proportional to the control signal. Further details of exemplary stimulus regimens may be found, for example, in U.S. Patent Application No. 60/584,730, which was previously incorporated by reference.

Control system 60 may be implanted in whole or in part. For example, the entire control system 60 may be carried externally by the patient utilizing transdermal connections to the sensor lead 82 and the control lead 72. Alternatively, control block 61 and driver 66 may be implanted with input device 64 and display 65 carried externally by the patient utilizing transdermal connections therebetween. As a further alternative, the transdermal connections may be replaced by cooperating transmitters/receivers to remotely communicate between components of control system 60 and/or sensor 80 and baroreflex/CRT device 70.

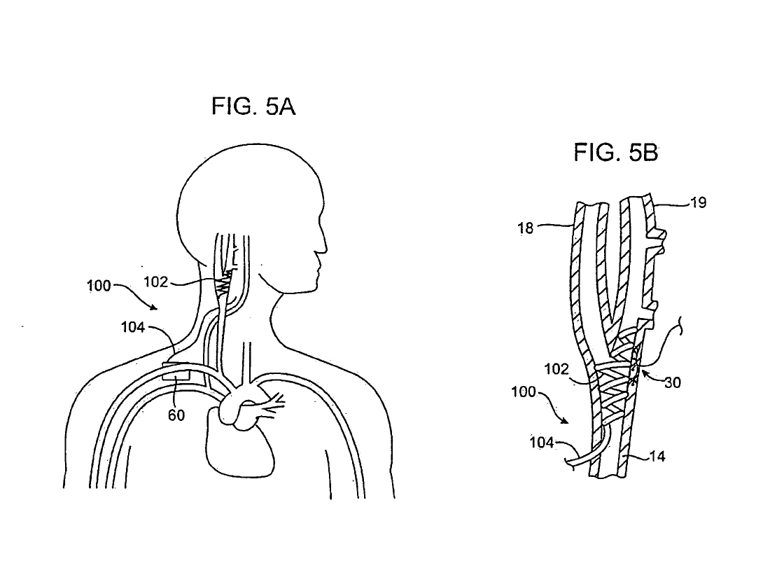

Referring now to FIGS. 5A and 5B, in one embodiment a baroreflex activation device 100 suitable for use in the present invention comprises an intravascular inflatable balloon. The inflatable balloon device 100 includes a helical balloon 102 which is connected to a fluid line 104. An example of a similar helical balloon is disclosed in U.S. Pat. No. 5,181,911 to Shturman, the entire disclosure of which is hereby incorporated by reference. The balloon 102 preferably has a helical geometry or any other geometry which allows blood perfusion therethrough. The fluid line 104 is connected to driver 66 of control system 60. In this embodiment, driver 66 comprises a pressure/vacuum source (i.e., an inflation device) which selectively inflates and deflates the helical balloon 102. Upon inflation, the helical balloon 102 expands, preferably increasing in outside diameter only, to mechanically activate baroreceptors 30 by stretching or otherwise deforming them and/or the vascular wall 40. Upon deflation, the helical balloon 102 returns to its relaxed geometry such that the vascular wall 40 returns to its nominal state. Thus, by selectively inflating the helical balloon 102, baroreceptors 30 adjacent thereto may be selectively activated.

As an alternative to pneumatic or hydraulic expansion utilizing a balloon, a mechanical expansion device (not shown) may be used to expand or dilate the vascular wall 40 and thereby mechanically activate baroreceptors 30. For example, the mechanical expansion device may comprise a tubular wire braid structure that diametrically expands when longitudinally compressed as disclosed in U.S. Pat. No. 5,222,971 to Willard et al., the entire disclosure of which is hereby incorporated by reference. The tubular braid may be disposed intravascularly and permits blood perfusion through the wire mesh. In this embodiment, driver 66 may comprise a linear actuator connected by actuation cables to opposite ends of the braid. When the opposite ends of the tubular braid are brought closer together by actuation of the cables, the diameter of the braid increases to expand the vascular wall 40 and activate baroreceptors 30.

For further details of exemplary baroreflex activation devices, reference may be made to U.S. Pat. Nos. 6,522,926 and 6,616,624, and U.S. patent application Ser. Nos. 09/964,079, 09/963,777, 09/963,991, 10/284,063, 10/453,678, 10/402,911, 10/402,393, 10/818,738, and 60/584,730, which were previously incorporated by reference.

Although the above description provides a complete and accurate representation of the invention, the present invention may be manifested in a variety of forms other than the specific embodiments described and contemplated herein. Accordingly, departures in form and detail may be made without departing from the scope and spirit of the present invention as described in the appended claims.

What is claimed is:

1. A neural stimulation system coupled to a living subject having a circulatory system, the neural stimulation system comprising:
a stimulation output circuit to deliver neural stimulation pulses;
an implantable reference signal sensor to sense a reference signal indicative of cardiac cycles each including a predetermined type timing reference event, the implantable reference signal sensor configured for placement external to the circulatory system; a reference event detection circuit coupled to the reference event sensor, the reference event detection circuit adapted to detect the predetermined type timing reference event; and
a stimulation control circuit coupled to the stimulation output circuit and the reference event detection circuit, the stimulation control circuit adapted to control the delivery of the neural stimulation pulses and including a synchronization module adapted to synchronize the delivery of the neural stimulation pulses to the predetermined type timing reference event.

2. The neural stimulation system of claim 1, further comprising an implantable housing adapted to contain at least the stimulation output circuit, the reference event detection circuit, and the stimulation control circuit.

3. The neural stimulation system of claim 2, wherein the reference signal comprises a subcutaneous electrocardiogram (ECG) signal, and wherein the implantable reference signal sensor comprises one or more subcutaneous electrodes adapted to sense a subcutaneous ECG signal.

4. The neural stimulation system of claim 1, wherein the reference signal comprises an acoustic signal indicative of heart sounds, and wherein the implantable reference signal sensor comprises an acoustic sensor to sense the acoustic signal.

5. The neural stimulation system of claim 1, wherein the reference signal comprises a signal indicative of a hemodynamic signal indicative of hemodynamic performance, and wherein the implantable reference signal sensor comprises a hemodynamic sensor to sense the hemodynamic signal.

6. The neural stimulation system of claim 1, wherein the reference event detection circuit comprises a signal processor adapted to extract the predetermined type timing reference event from the reference signal for each of the cardiac cycles.

7. The neural stimulation system of claim 6, wherein the synchronization module comprises a continuous synchronization module adapted to synchronize the delivery of the neural stimulation pulses to the predetermined type timing reference event consecutively for each of the cardiac cycles.

8. The neural stimulation system of claim 1, wherein the reference event detection circuit comprises a signal processor adapted to extract the predetermined type timing reference event from a segment of the reference signal associated with a plurality of cardiac cycles.

9. The neural stimulation system of claim 8, wherein the synchronization module comprises a periodic synchronization module adapted to synchronize the delivery of the neural stimulation pulses periodically for a cardiac cycle of a predetermined number of the cardiac cycles.

10. The neural stimulation system of claim 1, wherein stimulation control circuit comprises an offset interval generator to produce an offset interval starting with the predetermined type timing reference event and a pulse delivery controller adapted to start a delivery of a burst of a plurality of neural stimulation pulses when the offset interval expires.

11. A neural stimulation system, comprising:
a stimulation output circuit to deliver neural stimulation pulses; one or more implantable electrodes configured to sense a subcutaneous ECG signal;
a cardiac event detection circuit coupled to the one or more implantable electrodes, the cardiac event detection circuit adapted to detect predetermined type cardiac events from the subcutaneous ECG signal; and
a stimulation control circuit coupled to the stimulation output circuit and the cardiac event detection circuit, the stimulation control circuit adapted to control the delivery of the neural stimulation pulses and including a synchronization module adapted to synchronize the delivery of the neural stimulation pulses to the predetermined type cardiac events.

12. The neural stimulation system of claim 11, wherein the stimulation control circuit comprises an offset interval generator to produce an offset interval starting with one of the detected predetermined type cardiac events and a pulse delivery controller adapted to start a delivery of a burst of a plurality of neural stimulation pulses when the offset interval expires.

13. The neural stimulation system of claim 11, wherein the cardiac event detection circuit comprises an R-wave detector to detect ventricular depolarizations (R-waves).

14. The neural stimulation system of claim 11, wherein the cardiac event detection circuit comprises a P-wave detector to detect atrial depolarizations (P-waves).

15. The neural stimulation system of claim 14, wherein the P-wave detector comprises a signal averaging circuit to average the subcutaneous ECG signal over a plurality of cardiac cycles.

16. The neural stimulation system of claim 11, further comprising an implantable housing to house the stimulation output circuit, the cardiac event detection circuit, and the stimulation control circuit, and wherein the one or more implantable electrodes are incorporated onto the implantable housing.

17. The neural stimulation system of claim 16, further comprising an arrhythmia detection circuit to detect an arrhythmia from the subcutaneous ECG signal, and wherein the stimulation control circuit is adapted to withhold or adjust the delivery of the neural stimulation pulses when the arrhythmia is detected.

18. The neural stimulation system of claim 16, further comprising a cardiac parameter measurement circuit to measure one or more cardiac parameters from the subcutaneous ECG signal, and wherein the stimulation control circuit is adapted to adjust the delivery of the neural stimulation pulses based on the measured one or more cardiac parameters.

19. A neural stimulation system, comprising:
a stimulation output circuit to deliver neural stimulation pulses;
an acoustic sensor to sense an acoustic signal indicative of heart sounds;
a heart sound detection circuit coupled to the acoustic sensor, the heart sound detection circuit adapted to detect predetermined type heart sounds using the acoustic signal; and a stimulation control circuit coupled to the stimulation output circuit and the heart sound detection circuit, the stimulation control circuit adapted to control the delivery of the neural stimulation pulses and including a synchronization module adapted to synchronize the delivery of the neural stimulation pulses to the predetermined type heart sounds.

20. The neural stimulation system of claim 19, wherein the acoustic sensor comprises an implantable accelerometer.

21. The neural stimulation system of claim 20, wherein the stimulation control circuit comprises an offset interval generator to produce an offset interval starting with one of the predetermined type heart sounds and a pulse delivery controller to start a delivery of a burst of a plurality of neural stimulation pulses when the offset interval expires.

22. The neural stimulation system of claim 21, wherein the heart sound detection circuit comprises one or more of a first heart sound (S1) detector to detect S1, a second heart sound (S2) detector to detect S2, a third heart sound (S3) detector to detect S3, and a fourth heart sound (S4) detector to detect S4.

23. A neural stimulation system, comprising:
a stimulation output circuit to deliver neural stimulation pulses;
a hemodynamic sensor to sense a hemodynamic signal;
a hemodynamic event detection circuit coupled to the hemodynamic sensor, the hemodynamic event detection circuit adapted to detect predetermined type hemodynamic events using the hemodynamic signal; and
a stimulation control circuit coupled to the stimulation output circuit and the reference event detection circuit, the stimulation control circuit adapted to control the delivery of the neural stimulation pulses and including a synchronization module adapted to synchronize the delivery of the neural stimulation pulses to the predetermined type hemodynamic events.

24. The neural stimulation system of claim 23, further comprising a derivative calculator coupled to the hemodynamic event detection circuit, the derivative calculator adapted to produce a derivative hemodynamic signal by calculating a time derivative of the hemodynamic signal, and wherein the hemodynamic event detection circuit is adapted to detect the predetermined type hemodynamic events from the derivative hemodynamic signal.

25. The neural stimulation system of claim 23, wherein the stimulation control circuit comprises an offset interval generator to produce an offset interval starting with one of the detected predetermined type peaks and a pulse delivery controller to start a delivery of a burst of a plurality of neural stimulation pulses when the offset interval expires.

26. The neural stimulation system of claim 25, wherein the reference event detection circuit comprises a peak detector to detect predetermined type peaks in the hemodynamic signal.

27. The neural stimulation system of claim 25, wherein the hemodynamic sensor comprises a Doppler echocardiographic sensor to sense a hemodynamic signal indicative of blood flow.

28. The neural stimulation system of claim 25, wherein the hemodynamic sensor comprises a pressure sensor to sense a pressure signal indicative of blood pressure.

29. The neural stimulation system of claim 25, wherein the hemodynamic sensor comprises an impedance sensor to sense an impedance signal indicative of blood flow.

30. A method for operating a neural stimulation system coupled to a living subject having a circulatory system, the method comprising: sensing a reference signal indicative of cardiac cycles each including a predetermined type timing reference event using an implantable reference signal sensor placed external to the circulatory system; detecting the predetermined type timing reference event from the reference signal; and synchronizing a delivery of neural stimulation pulses to the detected predetermined type timing reference event.

31. The method of claim 30, wherein sensing the reference signal comprises sensing a subcutaneous ECG signal using implantable electrodes.

32. The method of claim 31, wherein detecting the predetermined type timing reference event from the reference signal comprises detecting an atrial depolarization (P-wave) from the subcutaneous ECG signal.

33. The method of claim 30, wherein sensing the reference signal comprises sensing an acoustic signal indicative of heart sounds, and detecting the predetermined type timing reference event from the reference signal comprises detecting predetermined type heart sounds from the acoustic signal.

34. The method of claim 30, wherein sensing the reference signal comprises sensing a hemodynamic signal indicative of blood flow or pressure.

35. The method of claim 34, wherein detecting the predetermined type timing reference event from the reference signal comprises detecting predetermined type peaks from the hemodynamic signal.

36. The method of claim 30, further comprising staring an offset interval with the detected timing reference event, and wherein synchronizing the delivery of neural stimulation pulses to the detected timing reference event comprises starting a delivery of a burst of a plurality of neural stimulation pulses when the offset interval expires.

37. The method of claim 36, wherein synchronizing the delivery of neural stimulation pulses comprises synchronizing the delivery of the neural stimulation pulses to the timing reference event of consecutive heart beats on a continuous basis.

38. The method of claim 36, wherein synchronizing the delivery of neural stimulation pulses comprises synchronizing the delivery of the neural stimulation pulses to the timing reference event of selected heart beats on a periodic basis.

39. The method of claim 36, wherein detecting the timing reference event from the reference signal comprises extracting the timing reference event from a segment of the reference signal recorded during a plurality of cardiac cycles.

40. A neural stimulation system coupled to a living subject having a circulatory system, the neural stimulation system comprising:
a stimulation output circuit to deliver neural stimulation pulses;
an implantable reference signal sensor to sense a reference signal indicative of cardiac cycles each including a predetermined type timing reference event, the implantable reference signal sensor configured for placement external to the circulatory system;
a reference event detection circuit coupled to the implantable reference signal sensor, the reference event detection circuit adapted to detect the predetermined type timing reference event; and
a stimulation control circuit coupled to the stimulation output circuit and the reference event detection circuit, the stimulation control circuit including:
an offset interval generator adapted to produce an offset interval starting with the predetermined type timing reference event; and
a pulse delivery controller adapted to start a delivery of a burst of a plurality of neural stimulation pulses when the offset interval expires.

41. The neural stimulation system of claim 40, further comprising an implantable housing adapted to contain at least the stimulation output circuit, the reference event detection circuit, and the stimulation control circuit.

42. The neural stimulation system of claim 41, wherein the reference signal comprises a subcutaneous electrocardiogram (ECG) signal, and wherein the implantable reference signal sensor comprises one or more subcutaneous electrodes adapted to sense a subcutaneous ECG signal.

43. The neural stimulation system of claim 40, wherein the reference event detection circuit comprises a signal processor adapted to extract the predetermined type timing reference event from the reference signal for each of the cardiac cycles.

44. The neural stimulation system of claim 43, wherein the synchronization module comprises a continuous synchronization module adapted to synchronize the delivery of the neural stimulation pulses to the predetermined type timing reference event consecutively for each of the cardiac cycles.

45. A neural stimulation system, comprising:
a stimulation output circuit to deliver neural stimulation pulses;
implantable electrodes configured to sense a subcutaneous ECG signal;
a cardiac event detection circuit coupled to the one or more implantable electrodes, the cardiac event detection circuit adapted to detect predetermined type cardiac events from the subcutaneous ECG signal;
a stimulation control circuit coupled to the stimulation output circuit and the cardiac event detection circuit, the stimulation control circuit adapted to control the delivery of the neural stimulation pulses and including a synchronization module adapted to synchronize the delivery of the neural stimulation pulses to the predetermined type cardiac events; and
an implantable housing adapted to house the stimulation output circuit, the cardiac event detection circuit, and the stimulation control circuit, wherein the implantable electrodes are incorporated onto the implantable housing.

46. The neural stimulation system of claim 45, wherein the stimulation control circuit comprises an offset interval generator to produce an offset interval starting with one of the detected predetermined type cardiac events and a pulse delivery controller adapted to start a delivery of a burst of a plurality of neural stimulation pulses when the offset interval expires.

47. The neural stimulation system of claim 45, wherein the cardiac event detection circuit comprises a P-wave detector to detect atrial depolarizations (P-waves).

48. The neural stimulation system of claim 47, wherein the P-wave detector comprises a signal averaging circuit to average the subcutaneous ECG signal over a plurality of cardiac cycles.
